(19) United States
(12) Patent Application Publication
(10) Pub. No.: US 2009/0291114 A1
(43) Pub. Date: Nov. 26, 2009

(54) OSTEOGENIC COMPOSITION COMPRISING A GROWTH FACTOR/AMPHIPHILIC POLYMER COMPLEX, A SOLUBLE CATION SALT AND AN ORGANIC SUPPORT

(75) Inventors: Remi Soula, Lyon (FR); Olivier Soula, Meyzieu (FR); Gerard Soula, Meyzieu (FR)

Correspondence Address: OLIFF & BERRIDGE, PLC, P.O. BOX 320850, ALEXANDRIA, VA, 22320-4850, US

(73) Assignee: ADOCIA, Lyon (FR)

(21) Appl. No.: 12/385,605

(22) Filed: Apr. 14, 2009

Related U.S. Application Data
(60) Provisional application No. 61/071,131, filed on Apr. 14, 2008.
(60) Provisional application No. 61/129,012, filed on May 30, 2008.
(60) Provisional application No. 61/129,616, filed on Jul. 8, 2008.
(60) Provisional application No. 61/193,216, filed on Nov. 6, 2008.

(30) Foreign Application Priority Data
Jul. 7, 2008 (FR) 0854621
Nov. 6, 2008 (FR) 0857560

(51) Int. Cl. A61K 38/18; A61F 2/00

(52) U.S. Cl. 424/423; 514/12

(57) ABSTRACT

The invention relates to an open implant constituted of an osteogenic composition comprising at least:
one osteogenic growth factor/amphiphilic anionic polysaccharide complex,
one soluble salt of a cation at least divalent, and
one organic support,
said organic support comprising no demineralized bone matrix.

In one embodiment, said implant is in the form of a lyophilizate.

It also relates to the method for the preparation thereof.

The present invention relates to the field of osteogenic formulations, and more particularly formulations of osteogenic proteins belonging to the bone morphogenetic protein, BMP, family.

Bone morphogenetic proteins (BMPs) are growth factors involved in osteoinduction mechanisms. BMPs, also known as osteogenic proteins (OPs), were initially characterized by Urist in 1965 (Urist M R. Science 1965; 150, 893). These proteins, isolated from cortical bone, have the ability to induce bone formation in a large number of animals (Urist M R. Science 1965; 150, 893).

BMPs are expressed in the form of propeptides which, after post-translational maturation, have a length of between 104 and 139 residues. They possess great sequence homology with respect to one another and have similar three-dimensional structures. In particular, they have six cysteine residues involved in intramolecular disulfide bridges forming a “cysteine knot” (Scheufler C. 2004 J. Mol. Biol. 1999; 287, 103; Schlunegger M P, J. Mol. Biol. 1993; 231, 445). Some of them have a 7^{th} cysteine also involved in an intermolecular disulfide bridge responsible for the formation of the dimer (Scheufler C. 2004 J. Mol. Biol. 1999; 287:103).

In their active form, BMPs assemble as homodimers, or even as heterodimers, as has been described by Israel et al. (Israel D I, Growth Factors. 1996; 13(3-4), 291). Dimeric BMPs interact with BMPR transmembrane receptors (Mundy et al. Growth Factors, 2004, 22 (4), 233). This recognition is responsible for an intracellular signaling cascade involving, in particular, Smad proteins, thus resulting in target gene activation or repression.

BMPs, with the exception of BMPs 1 and 3, play a direct and indirect role on the differentiation of mesenchymal cells, causing differentiation of the latter into osteoblasts (Cheng H., J. Bone and Joint Surgery, 2003, 85A 1544-1552). They also have chemotaxis properties and induce proliferation and differentiation.

Some recombinant human BMPs, and in particular rhBMP-2 and rhBMP-7, have clearly shown an ability to induce bone formation in vivo in humans and have been approved for some medical uses. Thus, recombinant human BMP-2, dibotermin alpha according to the international nonproprietary name, is formulated in products sold under the name InFUSE® in the United States and InductOs® in Europe. This product is prescribed in the fusion of lumbar vertebrae and bone regeneration in the tibia for “nonunion” fractures. In the case of InFUSE® for the fusion of lumbar vertebrae, the surgical procedure consists, first of all, in soaking a collagen sponge with a solution of rhBMP-2, and then in placing the sponge in a hollow cage, LT cage, preimplanted between the vertebrae.

Recombinant human BMP-7, eptotermin alpha according to the international nonproprietary name, has the same therapeutic indications as BMP-2 and constitutes the basis of two products: OP-1 Implant for open fractures of the tibia and OP-1 Putty for the fusion of lumbar vertebrae. OP-1 Implant is composed of a powder containing rhBMP-7 and collagen, to be taken up in a 0.9% saline solution. The paste obtained is subsequently applied to the fracture during a surgical procedure. OP-1 Puffy is in the form of two powders: one containing rhBMP-7 and collagen, the other containing carboxymethylatecellulose (CMC). During a surgical procedure, the solution of CMC is reconstituted with a 0.9% saline solution and mixed with the rhBMP-7 and the collagen. The resulting paste is applied to the site to be treated.

Patent application US2008/014197 describes an osteoinductive implant constituted of a support (scaffold) containing a mineral ceramic, of a solid membrane integrally bonded to the support and of an osteogenic agent. The support is preferably a collagen sponge. The mineral ceramic comprises a calcium derivative, preferably a water-insoluble mineral matrix such as biphasic calcium phosphate ([0024], p 2). The solid membrane integrally bonded to the implant should be impermeable so as to limit the entry of cells from the surrounding soft tissues and also to prevent the entry of inflammatory cells ([0030], p 3). The entry of these cells into the implant is described as possibly resulting in a reduction in bone growth and in failure of the treatment ([0007], p 1).

This invention is centered on the addition of a membrane to the implant in order to improve osteogenesis.

Patent application US2007/0254041 describes a device in the form of a sheet containing a demineralized bone matrix (or DBM), collagen particulate and a physically crosslinked polysaccharide matrix. This implant may, moreover, contain an osteogenic substance such as a growth factor. The physically crosslinked polysaccharide acts as a stabilizing agent for the particles of demineralized bone ([0026], p 3), said alginate-based polysaccharide being crosslinked through the addition of calcium chloride.

Patent application WO96/39203 describes a biocompatible, osteogenic composite material with physical strength. This osteoinductive material is composed of demineralized bone, it being possible for the osteoinduction to take place only in the presence of demineralized bone, or in the presence of protein extracts of demineralized bone, or in the presence of these two elements according to the authors (lines 2-5, p 2). A calcium salt or a mineral salt is added to this material. The mineral salt is described as possibly being sodium hydroxide, sodium chloride, magnesium chloride or magnesium hydroxide (lines 4-9, p 17). The calcium salt may or may not be a soluble salt (lines 20-21, p 17), and is preferably calcium hydroxide. The selection of the hydroxides of various cations, in particular calcium, to be added is justified by the effect of increasing the pH of the matrix, which favors increased collagen synthesis in this environment (lines 7-11, p 15).

This invention covers the formation of novel demineralized-bone-based implants, the physical and osteogenic properties of which would be improved by increasing the pH of the implant.

It has, moreover, been demonstrated that it is particularly advantageous to form complexes between a growth factor and a polymer with the aim of stabilizing it, of increasing its solubility and/or of increasing its activity.

Thus, in patent application FR0705536 in the name of the applicant, it was possible to demonstrate that the formation of a complex between BMP-2 and an amphiphilic polymer made it possible in particular to increase the solubility of this very hydrophobic protein that is relatively insoluble at physiological pH.

In patent application FR0705536, the applicant also demonstrated the increase in biological activity of BMP-2 in the presence of a dextran derivative functionalized with a hydrophobic substituent. In vitro, this BMP-2 complex appears to be superior in all respects to BMP-2 alone.

It remains, however, essential to find a formulation which makes it possible to improve the effectiveness of these BMP growth factors in order to be able, for example, to reduce the amounts to be administered.

This problem is common to many growth factor formulations since these proteins are, in general, used at doses which exceed the physiological doses by several orders of magnitude.

It is to the applicant's credit to have found a growth factor formulation which makes it possible to improve the activity of said growth factors through the addition of a solution of a soluble salt of a cation at least divalent to a hydrogel containing said growth factors, said soluble salt of a cation at least divalent potentiating the effect of the growth factor.

Surprisingly, this new formulation makes it possible to produce the same osteogenic effect with smaller amounts of growth factors.

The invention relates to an open implant constituted of an osteogenic composition comprising at least:
one osteogenic growth factor/amphiphilic anionic polysaccharide
complex,
one soluble salt of a cation at least divalent, and
one organic support,
said organic support comprising no demineralized bone matrix.

The term “open implant” is intended to mean an implant which comprises neither a membrane nor a shell capable of limiting or regulating exchanges with the tissues surrounding the implant and which is substantially homogeneous in terms of the constitution thereof.

The term “demineralized bone matrix” (or DBM) is intended to mean a matrix obtained by acid extraction of autologous bone, resulting in loss of the majority of the mineralized components but in preservation of the collagen proteins or noncollagen proteins, including the growth factors. Such a demineralized matrix may also be prepared in inactive form after extraction with chaotropic agents.

The term “organic support” is intended to mean a support constituted of an organic matrix and/or a hydrogel.

The term “organic matrix” is intended to mean a matrix constituted of crosslinked hydrogels and/or collagen.

The organic matrix is a hydrogel obtained by chemical crosslinking of polymer chains. The interchain covalent bonds defining an organic matrix. The polymers that may be used for making up an organic matrix are described in the review by Hoffman, entitled Hydrogels for biomedical applications (Adv. Drug Deliv. Rev, 2002, 43, 3-12).

In one embodiment, the matrix is selected from matrices based on sterilized, crosslinked, purified natural collagen.

The natural polymers such as collagen are extracellular matrix components which promote cell attachment, migration and differentiation. They have the advantage of being extremely biocompatible and are degraded by enzymatic digestion mechanisms. The collagen-based matrices are obtained from fibrillar collagen type I or IV, extracted from bovine or porcine tendon or bone. These collagens are first purified, before being crosslinked and then sterilized.

The organic supports according to the invention can be used as a mixture in order to obtain materials which may be in the form of a material with sufficient mechanical properties to be shaped or even molded, or else in the form of a “putty” or the collagen or a hydrogel plays a binder role.

Mixed materials can also be used, for example a matrix which combines collagen and inorganic particles and which may be in the form of a composite material with reinforced mechanical properties or else in the form of a “putty” or the collagen plays a binder role.

The inorganic materials that can be used comprise essentially ceramics based on calcium phosphate, such as hydroxyapatite (HA), tricalcium phosphate (TCP), biphasic calcium phosphate (BCP) or amorphous calcium phosphate (ACP), the main advantage of which is a chemical composition very close to that of bone. These materials have good mechanical properties and are immunologically inert. These materials may be in various forms, such as powders, granules or blocks. These materials have very different degradation rates, depending on their compositions; thus, hydroxyapatite degrades very slowly (several months) whereas tricalcium phosphate degrades more rapidly (several weeks). Biphasic calcium phosphates were developed for this purpose, since they have intermediate resorption rates. These inorganic materials are known to be principally osteoconductive.

The term “hydrogel” is intended to mean a hydrophilic three-dimensional network of polymer capable of adsorbing a large amount of water or of biological fluids (Peppas et al., Eur. J. Pharm. Biopharm. 2000, 50, 27-46). Such a hydrogel is constituted of physical interactions and is not therefore obtained by chemical crosslinking of the polymer chains.

Among these polymers may be found synthetic polymers and natural polymers. The polysaccharides forming hydrogels are described, for example, in the article entitled: Polysaccharide hydrogels for modified release formulations (Coviello et al. J. Control. Release, 2007, 119, 5-24).

In one embodiment, the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

In one embodiment, the polymer forming a hydrogel is selected from the group of natural polymers, among which are hyaluronic acid, keratan, pullulan, pectin, dextran, cellulose and cellulose derivatives, alginic acid, xanthan, carrageenan, chitosan, chondroitin, collagen, gelatin, polylysine and fibrin, and biologically acceptable salts thereof.

In one embodiment, the natural polymer is selected from the group of polysaccharides forming hydrogels, among which are hyaluronic acid, alginic acid, dextran, pectin, cellulose and its derivatives, pullulan, xanthan, carrageenan, chitosan and chondroitin, and biologically acceptable salts thereof.

In one embodiment, the natural polymer is selected from the group of polysaccharides forming hydrogels, among which are hyaluronic acid and alginic acid, and biologically acceptable salts thereof.

The term “amphiphilic polysaccharide” is intended to mean a polysaccharide selected from the group of polysaccharides functionalized with hydrophobic derivatives.

These polysaccharides are constituted predominantly of glycosidic linkages of (1,4) and/or (1,3) and/or (1,2) type. They may be neutral, i.e. not carrying acid functions, or anionic and carrying acid functions.

They are functionalized with at least one tryptophan derivative, denoted Trp:
said tryptophan derivative being grafted or bonded to the polysaccharides by coupling with an acid function, it being possible for said acid function to be an acid function of an anionic polysaccharide and/or an acid function carried by a linker arm R linked to the polysaccharide by a function F, said function F resulting from the coupling between the linker arm R and a function —OH of the neutral or anionic polysaccharide,
   F being either an ester function, a thioester function, an amide function, a carbonate function, a carbamate function, an ether function, a thioether function or an amine function,
   R being an optionally branched and/or unsaturated chain containing between 1 and 18 carbons, comprising one or more heteroatoms, such as O, N and/or S, and having at least one acid function,
Trp being a residue of an L- or D-tryptophan derivative, produced from the coupling between the amine of the tryptophan and the at least one acid carried by the R group and/or one acid carried by the anionic polysaccharide.

According to the invention, the polysaccharide comprising predominantly glycosidic linkages of (1,4), (1,3) and/or (1,2) type, functionalized with at least one tryptophan derivative, may correspond to general formula I below:

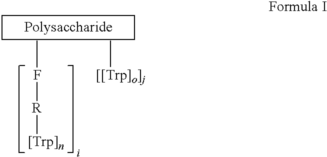

the polysaccharide being constituted predominantly of glycosidic linkages of (1,4) and/or (1,3) and/or (1,2) type,
F resulting from the coupling between the linker arm R and a function —OH of the neutral or anionic polysaccharide, being either an ester function, a thioester function, an amide function, a carbonate function, a carbamate function, an ether function, a thioether function or an amine function,
R being an optionally branched and/or unsaturated chain containing between 1 and 18 carbons, comprising one or more heteroatoms, such as O, N and/or S, and having at least one acid function,
Trp being a residue of an L- or D-tryptophan derivative, produced from the coupling between the amine of the tryptophan derivative and the at least one acid carried by the R group and/or one acid carried by the anionic polysaccharide,
   n is the molar fraction of the Trp-substituted Rs and is between 0.05 and 0.7,
   o is the molar fraction of the acid functions of the Trp-substituted polysaccharides and is between 0.05 and 0.7,
   i is the molar fraction of acid functions carried by the R group per saccharidic unit and is between 0 and 2,
   j is the molar fraction of acid functions carried by the anionic polysaccharide per saccharidic unit and is between 0 and 1,
   (i+j) is the molar fraction of acid functions per saccharidic unit and is between 0.1 and 2,
      when R is not substituted with Trp, then the acid(s) of the R group is (are) a cation carboxylate or cation carboxylates, the cation being a cation of an alkali metal, preferably such as Na or K,
      when the polysaccharide is an anionic polysaccharide, when one or more acid function(s) of the polysaccharide is (are) not substituted with Trp, then it (they) is (are) salified with a cation, the cation being an alkali metal cation, preferably such as Na⁺ or K⁺,

said polysaccharides being amphiphilic at neutral pH.

In one embodiment, F is either an ester, a carbonate, a carbamate or an ether.

In one embodiment, the polysaccharide is constituted predominantly of glycosidic linkages of (1,4) type.

In one embodiment, the polysaccharide constituted predominantly of glycosidic linkages of (1,4) type is selected from the group constituted of pullulan, alginate, hyaluronan, xylan, galacturonan or a water-soluble cellulose.

In one embodiment, the polysaccharide is a pullulan.

In one embodiment, the polysaccharide is an alginate.

In one embodiment, the polysaccharide is a hyaluronan.

In one embodiment, the polysaccharide is a xylan.

In one embodiment, the polysaccharide is a galacturonan.

In one embodiment, the polysaccharide is a water-soluble cellulose.

In one embodiment, the polysaccharide is constituted predominantly of glycosidic linkages of (1,3) type.

In one embodiment, the polysaccharide constituted predominantly of glycosidic linkages of (1,3) type is a curdlan.

In one embodiment, the polysaccharide is constituted predominantly of glycosidic linkages of (1,2) type.

In one embodiment, the polysaccharide constituted predominantly of glycosidic linkages of (1,2) type is an inulin.

In one embodiment, the polysaccharide is constituted predominantly of glycosidic linkages of (1,4) and (1,3) type.

In one embodiment, the polysaccharide constituted predominantly of glycosidic linkages of (1,4) and (1,3) type is a glucan.

In one embodiment, the polysaccharide is constituted predominantly of glycosidic linkages of (1,4) and (1,3) and (1,2) type.

In one embodiment, the polysaccharide constituted predominantly of glycosidic linkages of (1,4) and (1,3) and (1,2) type is mannan.

In one embodiment, the polysaccharide according to the invention is characterized in that the R group is selected from the following groups:

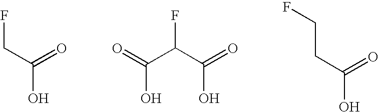

or the alkali-metal cation salts thereof.

In one embodiment, the polysaccharide according to the invention is characterized in that the tryptophan derivative is selected from the group constituted of tryptophan, tryptophanol, tryptophanamide and 2-indole ethylamine, and the alkali-metal cation salts thereof.

In one embodiment, the polysaccharide according to the invention is characterized in that the tryptophan derivative is selected from the tryptophan esters of formula II:

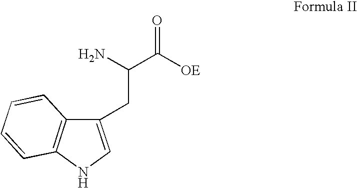

E being a group that may be:
a linear or branched (C1-C8) alkyl;
a linear or branched (C6-C20) alkylaryl or arylalkyl.

The polysaccharide may have a degree of polymerization m of between 10 and 10 000.

In one embodiment, it has a degree of polymerization m of between 10 and 1000.

In another embodiment, it has a degree of polymerization m of between 10 and 500.

In one embodiment, the polysaccharides are selected from the group of dextrans functionalized with hydrophobic amino acids such as tryptophan and the tryptophan derivatives as described in application FR 07/02316.

According to the invention, the functionalized dextran may correspond to general formula III below:

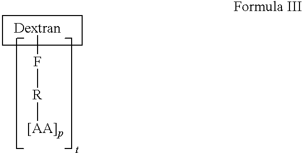

R being an optionally branched and/or unsaturated chain containing between 1 and 18 carbons, comprising one or more heteroatoms, such as O, N and/or S, and having at least one acid function,
F resulting from the coupling between the linker arm R and a function —OH of the neutral or anionic polysaccharide, being either an ester function, a thioester function, an amide function, a carbonate function, a carbamate function, an ether function, a thioether function or an amine function,
AA being a hydrophobic L- or D-amino acid residue produced from the coupling between the amine of the amino acid and an acid carried by the R group,
   t is the molar fraction of F—R-[AA]n substituent per glycosidic unit and is between 0.1 and 2,
   p is the molar fraction of the M-substituted R groups and is between 0.05 and 1.

When R is not substituted with AA, then the acid(s) of the R group is (are) a cation carboxylate or cation carboxylates, the cation being an alkali metal cation, preferably such as Na⁺ or K⁺,

said dextran being amphiphilic at neutral pH.

In one embodiment, the alkali metal cation is Na⁺.

In one embodiment, F is either an ester, a carbonate, a carbamate or an ether.

In one embodiment, the polysaccharide according to the invention is a carboxymethylate dextran of formula IV:

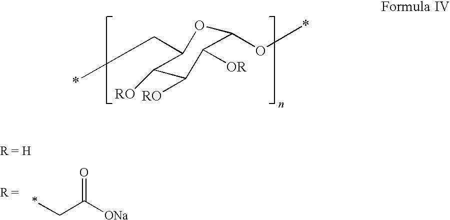

or the corresponding acid.

In one embodiment, the polysaccharide according to the invention is a monosuccinic ester of dextran of formula V:

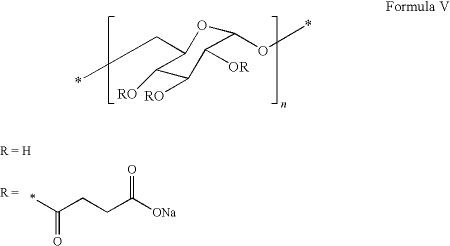

or the corresponding acid.

In one embodiment, the polysaccharide according to the invention is characterized in that the R group is selected from the following groups:

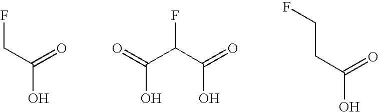

or the alkali-metal cation salts thereof.

In one embodiment, the dextran according to the invention is characterized in that the hydrophobic amino acid is selected from tryptophan derivatives such as tryptophan, tryptophanol, tryptophanamide and 2-indole ethylamine, and the alkali-metal cation salts thereof.

In one embodiment, the dextran according to the invention is characterized in that the tryptophan derivatives are selected from the tryptophan esters of formula II as defined above.

In one embodiment, the dextran according to the invention is a tryptophan-modified carboxymethylate dextran of formula VI:

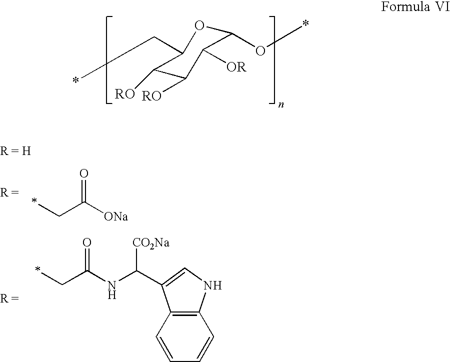

In one embodiment, the dextran according to the invention is a tryptophan-modified monosuccinic ester of dextran of formula VII:

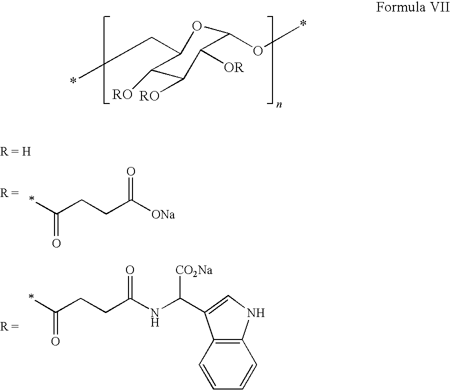

In one embodiment, the dextran according to the invention is characterized in that the hydrophobic amino acid is selected from phenylalanine, leucine, isoleucine and valine, and the alcohol, amide or decarboxylated derivatives thereof.

In one embodiment, the dextran according to the invention is characterized in that the phenylalanine, leucine, isoleucine and valine derivatives are selected from the esters of these amino acids, of formula VIII:

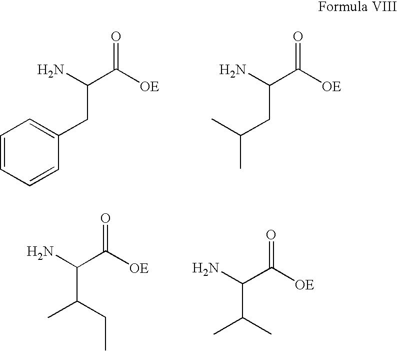

E being defined as above.

In one embodiment, the dextran according to the invention is characterized in that the hydrophobic amino acid is phenylalanine, or the alcohol, amide or decarboxylated derivatives thereof.

The dextran may have a degree of polymerization m of between 10 and 10 000.

In one embodiment, it has a degree of polymerization m of between 10 and 1000.

In another embodiment, it has a degree of polymerization m of between 10 and 500.

In one embodiment, the polysaccharides are selected from the group of polysaccharides comprising carboxyl functional groups such as those described in application FR 08/05506, at least one of which is substituted with a hydrophobic alcohol derivative, denoted Ah:
said hydrophobic alcohol (Ah) being grafted or bonded to the anionic polysaccharide by a coupling arm R, said coupling arm being bonded to the anionic polysaccharide by a function F′, said function F′ resulting from the coupling between the amine function of the linker arm R and a carboxyl function of the anionic polysaccharide, and said coupling arm being bonded to the hydrophobic alcohol by a function G resulting from the coupling between a carboxyl, isocyanate, thioacid or alcohol function of the coupling arm and a function of the hydrophobic alcohol, the unsubstituted carboxyl functions of the anionic polysaccharide being in the form of a cation carboxylate, the cation being an alkali metal cation, preferably such as Na⁺ or K⁺,
   F′ being an amide function,
   G being either an ester function, a thioester function, a carbonate function or a carbamate function,
   R being an optionally branched and/or unsaturated chain containing between 1 and 18 carbons, optionally comprising one or more heteroatoms, such as O, N and/or S, and having at least one acid function,
Ah being a residue of a hydrophobic alcohol, produced from the coupling between the hydroxyl function of the hydrophobic alcohol and at least one electrophilic function carried by the R group,
   said polysaccharide comprising carboxyl functional groups being amphiphilic at neutral pH.

The polysaccharide comprising carboxyl functional groups partially substituted with hydrophobic alcohols is selected from the polysaccharides comprising carboxyl functional groups of general formula IX:

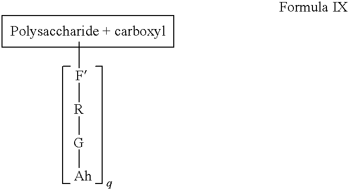

in which q is the molar fraction of the F—R-G-Ah-substituted carboxyl functions of the polysaccharide and is between 0.01 and 0.7,
F′, R, G and Ah corresponding to the definitions given above, and when the carboxyl function of the polysaccharide is not substituted with F′—R-G-Ah, then the carboxyl functional group(s) of the polysaccharide is (are) a cation carboxylate or cation carboxylates, the cation being an alkali metal cation, preferably such as Na⁺ or K⁺.

In one embodiment, the polysaccharides comprising carboxyl functional groups are polysaccharides that naturally carry carboxyl functional groups and are selected from the group constituted of alginate, hyaluronan and galacturonan.

In one embodiment, the polysaccharides comprising carboxyl functional groups are synthetic polysaccharides obtained from polysaccharides that naturally comprise carboxyl functional groups or from neutral polysaccharides onto which at least 15 carboxyl functional groups per 100 saccharidic units have been grafted, of general formula X:

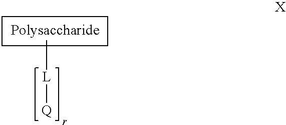

the natural polysaccharides being selected from the group of polysaccharides constituted predominantly of glycosidic linkages of (1,6) and/or (1,4) and/or (1,3) and/or (1,2) type,
L being a link resulting from the coupling between the linker arm Q and a function —OH of the polysaccharide and being either an ester function, a thioester function, a carbonate function, a carbamate function or an ether function,
r is the molar fraction of the substituents L-Q per saccharidic unit of the polysaccharide,

Q being an optionally branched and/or unsaturated chain containing between 1 and 18 carbons, comprising one or more heteroatoms, such as O, N and/or S, and comprising at least one carboxyl functional group, —CO₂H.

In one embodiment, the polysaccharide is constituted predominantly of glycosidic linkages of (1,6) type.

In one embodiment, the polysaccharide constituted predominantly of glycosidic linkages of (1,6) type is dextran.

In one embodiment, the polysaccharide is constituted predominantly of glycosidic linkages of (1,4) type.

In one embodiment, the polysaccharide constituted predominantly of glycosidic linkages of (1,4) type is selected from the group constituted of pullulan, alginate, hyaluronan, xylan, galacturonan or a water-soluble cellulose.

In one embodiment, the polysaccharide is a pullulan.

In one embodiment, the polysaccharide is an alginate.

In one embodiment, the polysaccharide is a hyaluronan.

In one embodiment, the polysaccharide is a xylan.

In one embodiment, the polysaccharide is a galacturonan.

In one embodiment, the polysaccharide is a water-soluble cellulose.

In one embodiment, the polysaccharide is constituted predominantly of glycosidic linkages of (1,3) type.

In one embodiment, the polysaccharide constituted predominantly of glycosidic linkages of (1,3) type is a curdlan.

In one embodiment, the polysaccharide is constituted predominantly of glycosidic linkages of (1,2) type.

In one embodiment, the polysaccharide constituted predominantly of glycosidic linkages of (1,2) type is an inulin.

In one embodiment, the polysaccharide is constituted predominantly of glycosidic linkages of (1,4) and (1,3) type.

In one embodiment, the polysaccharide constituted predominantly of glycosidic linkages of (1,4) and (1,3) type is a glucan.

In one embodiment, the polysaccharide is constituted predominantly of glycosidic linkages of (1,4) and (1,3) and (1,2) type.

In one embodiment, the polysaccharide constituted predominantly of glycosidic linkages of (1,4) and (1,3) and (1,2) type is mannan.

In one embodiment, the polysaccharide according to the invention is characterized in that the Q group is selected from the following groups:

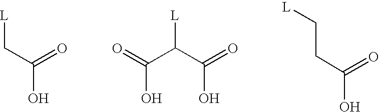

In one embodiment, r is between 0.1 and 2.

In one embodiment, r is between 0.2 and 1.5.

In one embodiment, the R group according to the invention is characterized in that it is selected from amino acids.

In one embodiment, the amino acids are selected from alpha-amino acids.

In one embodiment, the alpha-amino acids are selected from natural alpha-amino acids.

In one embodiment, the natural alpha-amino acids are selected from leucine, alanine, isoleucine, glycine, phenylalanine, tryptophan and valine.

In one embodiment, the hydrophobic alcohol is selected from fatty alcohols.

In one embodiment, the hydrophobic alcohol is selected from alcohols constituted of an unsaturated or saturated alkyl chain containing from 4 to 18 carbons.

In one embodiment, the fatty alcohol is selected from myristyl alcohol, cetyl alcohol, stearyl alcohol, cetearyl alcohol, butyl alcohol, oleyl alcohol and lanolin.

In one embodiment, the hydrophobic alcohol is selected from cholesterol derivatives.

In one embodiment, the cholesterol derivative is cholesterol.

In one embodiment, the hydrophobic alcohol Ah is selected from tocopherols.

In one embodiment, the tocopherol is alpha-tocopherol.

In one embodiment, the alpha-tocopherol is the racemic mixture of alpha-tocopherol.

In one embodiment, the hydrophobic alcohol is selected from alcohols carrying an aryl group.

In one embodiment, the alcohol carrying an aryl group is selected from benzyl alcohol and phenethyl alcohol.

The polysaccharide may have a degree of polymerization m of between 10 and 10 000.

In one embodiment, it has a degree of polymerization m of between 10 and 1000.

In another embodiment, it has a degree of polymerization m of between 10 and 500.

In one embodiment, said composition is in the form of a lyophilizate.

In one embodiment, the soluble salt of a cation at least divalent is a soluble salt of a divalent cation selected from calcium, magnesium or zinc cations.

In one embodiment, the soluble salt of a cation at least divalent is a soluble calcium salt.

The term “soluble salt of a cation at least divalent” is intended to mean a salt of which the solubility is greater than or equal to 5 mg/ml, preferably 10 mg/ml, preferably 20 mg/ml.

In one embodiment, the soluble divalent-cation salt is a calcium salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is a magnesium salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is a zinc salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is calcium chloride.

In one embodiment, the soluble cation salt is a soluble multivalent-cation salt.

The term “multivalent cations” is intended to mean species carrying more than two positive charges, such as iron, aluminum, cationic polymers such as polylysine, spermine, protamine or fibrin.

The term “osteogenic growth factor”, or “BMP”, alone or in combination is intended to mean a BMP selected from the group of therapeutically active BMPs (bone morphogenetic proteins).

More particularly, the osteogenic proteins are selected from the group constituted of BMP-2 (dibotermin alpha), BMP-4, BMP-7 (eptotermin alpha), BMP-14 and GDF-5.

In one embodiment, the osteogenic protein is BMP-2 (dibotermin alpha).

In one embodiment, the osteogenic protein is GDF-5.

The BMPs used are recombinant human BMPs obtained according to the techniques known to those skilled in the art or purchased from suppliers such as, for example, the company Research Diagnostic Inc. (USA).

In one embodiment, the hydrogel may be prepared just before implantation.

In one embodiment, the hydrogel may be prepared and stored in a prefilled syringe in order to be subsequently implanted.

In one embodiment, the hydrogel may be prepared by rehydration of a lyophilizate just before implantation or may be implanted in dehydrated form.

Lyophilization is a water sublimation technique enabling dehydration of the composition. This technique is commonly used for the storage and stabilization of proteins.

The rehydration of a lyophilizate is very rapid and enables a ready-to-use formulation to be easily obtained, it being possible for said formulation to be rehydrated before implantation, or implanted in its dehydrated form, the rehydration then taking place, after implantation, through the contact with the biological fluids.

In addition, it is possible to add other proteins, and in particular angiogenic growth factors such as PDGF, VEGF or FGF, to these osteogenic growth factors.

The invention therefore relates to a composition according to the invention, characterized in that it further comprises angiogenic growth factors selected from the group constituted of PDGF, VEGF or FGF.

The osteogenic compositions according to the invention are used by implantation, for example, for filling bone defects, for performing vertebral fusions or maxillofacial reconstructions, or for treating an absence of fracture consolidation (pseudarthrosis).

In these various therapeutic uses, the size of the matrix and the amount of osteogenic growth factor depend on the volume of the site to be filled.

In one embodiment, the solutions of anionic polysaccharide have concentrations of between 0.1 mg/ml and 100 mg/ml, preferably 1 mg/ml to 75 mg/ml, more preferably between 5 and 50 mg/ml.

In one embodiment, for a vertebral implant, the doses of osteogenic growth factor will be between 0.05 mg and 8 mg, preferably between 0.1 mg and 4 mg, more preferably between 0.1 mg and 2 mg, whereas the doses commonly accepted in the literature are between 8 and 12 mg.

In one embodiment, for a vertebral implant, the doses of angiogenic growth factor will be between 0.05 mg and 8 mg, preferably between 0.1 mg and 4 mg, more preferably between 0.1 mg and 2 mg.

As regards the uses in maxillofacial reconstruction or in the treatment of pseudarthrosis, for example, the doses administered will be less than 1 mg.

In one embodiment, the solutions of divalent cation have concentrations of between 0.01 and 1 M, preferably between 0.05 and 0.2 M.

In one embodiment, the solutions of anionic polysaccharide have concentrations of between 0.1 mg/ml and 100 mg/ml, preferably 1 mg/ml to 75 mg/ml, more preferably between 5 and 50 mg/ml.

The invention also relates to the method for preparing an implant according to the invention, which comprises at least the following steps:
a) providing a solution comprising an osteogenic growth factor/anionic polysaccharide complex, and an organic matrix and/or a hydrogel,
b) adding the solution containing the complex to the organic matrix and/or to the hydrogel, and optionally homogenizing the mixture,
c) adding a solution of a soluble salt of a cation at least divalent to the implant obtained in b),
d) optionally carrying out the lyophilization of the implant obtained in step c).

The invention also relates to the method for preparing an implant according to the invention, which comprises at least the following steps:
a) providing a solution comprising an osteogenic growth factor/amphiphilic anionic polysaccharide complex, and an organic matrix and/or a hydrogel,
b) adding a solution of a soluble salt of a cation at least divalent to the organic matrix and/or to the hydrogel a),
c) adding the solution containing the growth factor to the organic matrix and/or to the hydrogel obtained in b) and optionally homogenizing the mixture,
d) optionally carrying out the lyophilization of the implant obtained in step c).

In one embodiment, the organic matrix is a matrix constituted of crosslinked hydrogels and/or collagen.

In one embodiment, the matrix is selected from matrices based on sterilized, preferably crosslinked, purified natural collagen.

In one embodiment, in step a), the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

In one embodiment, in step a), the polymer forming a hydrogel, which may be crosslinked or noncrosslinked, is selected from the group of natural polymers, among which are hyaluronic acid, keratan, pectin, dextran, cellulose and cellulose derivatives, alginic acid, xanthan, carrageenan, chitosan, chondroitin, collagen, gelatin, polylysine and fibrin, and biologically acceptable salts thereof.

In one embodiment, the natural polymer is selected from the group of polysaccharides forming hydrogels, among which are hyaluronic acid, alginic acid, dextran, pectin, cellulose and its derivatives, pullulan, xanthan, carrageenan, chitosan and chondroitin, and biologically acceptable salts thereof.

In one embodiment, in step a), the natural polymer is selected from the group of polysaccharides forming hydrogels, among which are hyaluronic acid and alginic acid, and biologically acceptable salts thereof.

In one embodiment, in step b) or c), the solution of a soluble salt of a cation at least divalent is a divalent-cation solution.

In one embodiment, the soluble divalent-cation salts are calcium salts, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is calcium chloride.

In one embodiment, the soluble divalent-cation salts are magnesium salts, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salts are zinc salts, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, in step b) or c), the solution of a soluble salt of a cation at least divalent is a multivalent-cation solution.

In one embodiment, the multivalent cations are selected from the group constituted of the multivalent cations of iron, aluminum or cationic polymers such as polylysine, spermine, protamine or fibrin.

In one embodiment, in step a), a solution of a nonosteogenic growth factor is also provided.

The invention also relates to the use of the composition according to the invention, as a bone implant.

In one embodiment, said composition may be used in combination with a prosthetic device of the vertebral prosthesis or vertebral fusion cage type.

It also relates to the therapeutic and surgical methods using said composition in bone reconstruction.

The invention is illustrated by the following examples.

### EXAMPLE 1

### Preparation of a Sodium Carboxymethylate Dextran Modified with the Sodium Salt of L-Tryptophan

Polymer 1 is a sodium carboxymethylate dextran modified with the sodium salt of L-tryptophan, obtained from a dextran having a weight-average molar mass of 40 kg/mol, i.e. a degree of polymerization of 154 (Pharmacosmos), according to the method described in patent application FR07.02316. The molar fraction of sodium carboxymethylate derivatives, which may or may not be modified with tryptophan, i.e. t in formula I, is 1.03. The molar fraction of sodium carboxymethylate derivatives modified with tryptophan, i.e. p in formula II, is 0.36.

### EXAMPLE 2

### Preparation of a Sodium Carboxymethylate Dextran Modified with the Ethyl Ester of L-Tryptophan

Polymer 2 is a sodium carboxymethylate dextran modified with the ethyl ester of L-tryptophan, obtained from a dextran having a weight-average molar mass of 40 kg/mol, i.e. a degree of polymerization of 154 (Pharmacosmos), according to the method described in patent application FR07.02316. The molar fraction of sodium carboxymethylate, which may or may not be modified with the ethyl ester of tryptophan, i.e. t in formula III, is 1.07. The molar fraction of sodium carboxymethylate modified with the ethyl ester of tryptophan, i.e. p in formula III, is 0.49.

### EXAMPLE 3

### Preparation of a Sodium Carboxymethylate Dextran Modified with the Decyl Ester of L-Glycine

Polymer 3 is a sodium carboxymethylate dextran modified with the decyl ester of L-glycine, obtained from a dextran having a weight-average molar mass of 40 kg/mol, i.e. a degree of polymerization of 154 (Pharmacosmos), according to the method described in patent application FR08.05506. The molar fraction of sodium carboxymethylate, which may or may not be modified with the decyl ester of L-glycine, i.e. r in formula X, is 1.04. The molar fraction of sodium carboxymethylate modified with the decyl ester of L-glycine, i.e. q in formula IX, is 0.09.

### EXAMPLE 4

### Preparation of a Sodium Carboxymethylate Dextran Modified with the Octanoic Ester of L-Phenylalanine

Polymer 4 is a sodium carboxymethylate dextran modified with the octanoic ester of L-phenylalanine, obtained from a dextran having a weight-average molar mass of 40 kg/mol, i.e. a degree of polymerization of 154 (Pharmacosmos), according to the method described in patent application FR08.05506. The molar fraction of sodium carboxymethylate, which may or may not be modified with the octanoic ester of L-phenylalanine, i.e. r in formula X, is 1.07. The molar fraction of sodium carboxymethylate modified with the octanoic ester of L-phenylalanine, i.e. q in formula IX, is 0.08.

### EXAMPLE 5

### Preparation of the rhGDF-5/Polymer 3 Complex

Formulation 1: 50 μl of a solution of rhGDF-5 at 2.0 mg/ml in 5 mM HCl are mixed with 50 μl of a solution of polymer 3 at 61.1 mg/ml. The polymer solution is buffered with 20 mM of phosphate (pH of 7.2). The solution of GDF-5/polymer 3 complex is at pH 6.4 and contains 10 mM of phosphate. The GDF-5/polymer 3 molar ratio is 1/20. This solution is finally filtered through 0.22 μm. The final solution is clear and is characterized by dynamic light scattering. The majority of the objects present measure less than 10 nm.

### EXAMPLE 6

### Preparation of the rhGDF-5/Polymer 4 Complex

Formulation 2: 679 μl of a solution of rhGDF-5 at 3.7 mg/ml in 10 mM HCl are mixed with 1821 μl of a solution of polymer 4 at 42.3 mg/ml (pH of 7.3). The solution of GDF-5/polymer 4 complex is at pH 6.5 and contains 1 mg/ml of GDF-5 and 30.8 mg/ml of polymer 4. The GDF-5/polymer 4 molar ratio is 1/20. This solution is finally filtered through 0.22 μm. The final solution is clear and is characterized by dynamic light scattering. The majority of the objects present measure less than 10 nm.

### EXAMPLE 7

### Preparation of Collagen Sponge/rhBMP-2 Implants

Implant 1: 40 μl of a solution of rhBMP-2 at 0.05 mg/ml are introduced sterilely into a Helistat type sterile 200 mm³ crosslinked collagen sponge (Integra LifeSciences, Plainsboro, N.J.). The solution is left to incubate for 30 minutes in the collagen sponge before use. The dose of BMP-2 is 2 μg.

Implant 2: It is prepared like implant 1, with 40 μl of a solution of rhBMP-2 at 0.5 mg/ml. The dose of BMP-2 is 20 μg.

### EXAMPLE 8

### Preparation of the rhBMP-2/Polymer 1 Complex

Formulation 3: 50 μl of a solution of rhBMP-2 at 0.15 mg/ml are mixed with 100 μl of a solution of polymer 1 at 37.5 mg/ml. The solutions of rhBMP-2 and of polymer 1 are buffered at pH 7.4. This solution is left to incubate for two hours at 40° C. and filtered sterilely through 0.22 μm.

Formulation 4: It is prepared like formulation 3, by mixing 50 μl of a solution of rhBMP-2 at 1.5 mg/ml with 100 μl of a solution of polymer 1 at 37.5 mg/ml.

### EXAMPLE 9

### Preparation of Implants of Collagen Sponge/BMP-2/Polymer 1 Complex in the Presence of Calcium Chloride, Which are Lyophilized

Implant 3: 40 μl of formulation 4 are introduced into a Helistat type sterile 200 mm³ crosslinked collagen sponge (Integra LifeSciences, Plainsboro, N.J.). The solution is left to incubate for 30 minutes in the collagen sponge before adding 100 μl of a solution of calcium chloride at a concentration of 18.3 mg/ml. After 15 minutes, the sponge is ready for use. The dose of BMP-2 is 20 μg.

### EXAMPLE 10

### Preparation of Implants of Collagen Sponge/BMP-2/Polymer 1 Complex in the Presence of Calcium Chloride, which are Lyophilized

Implant 4: 40 μl of formulation 3 are introduced into a Helistat type sterile 200 mm³ crosslinked collagen sponge (Integra LifeSciences, Plainsboro, N.J.). The solution is left to incubate for 30 minutes in the collagen sponge before adding 100 μl of a solution of calcium chloride at a concentration of 18.3 mg/ml. The sponge is then subsequently frozen and lyophilized sterilely. The dose of BMP-2 is 2 μg.

Implant 5: It is prepared like implant 4, with 40 μl of formulation 4. The dose of BMP-2 is 20 μg.

### EXAMPLE 11

### Evaluation of the Osteoinductive Capacity of the Various Formulations

The objective of this study is to demonstrate the osteoinductive capacity of the various formulations in a model of ectopic bone formation in the rat. Male rats weighing 150 to 250 g (Sprague Dawley OFA-SD, Charles River Laboratories France, B.P. 109, 69592 l'Arbresle) are used for this study.

An analgesic treatment (buprenorphine, Temgesic®, Pfizer, France) is administered before the surgical procedure. The rats are anesthetized by inhalation of an O₂-isoflurane mixture (1-4%). The fur is removed by shaving over a wide dorsal area. The skin of this dorsal area is disinfected with a solution of povidone-iodine (Vetedine® solution, Vetoquinol, France).

Paravertebral incisions of approximately 1 cm are made in order to free the right and left dorsal paravertebral muscles. Access to the muscles is made by transfascial incision. Each of the implants is placed in a pocket in such a way that no compression can be exerted thereon. Four implants are implanted per rat (two implants per site). The implant opening is then sutured using a polypropylene thread (Prolene 4/0, Ethicon, France). The skin is re-closed using a nonabsorbable suture. The rats are then returned to their respective cages and kept under observation during their recovery.

At 21 days, the animals are anesthetized with an injection of tiletamine-zolazepam (ZOLETIL® 25-50 mg/kg, 1M, VIRBAC, France).

The animals are then sacrificed by euthanasia, by injecting a dose of pentobarbital (DOLETHAL®, VETOQUINOL, France). A macroscopic observation of each site is then carried out; any sign of local intolerance (inflammation, necrosis, hemorrhage) and the presence of bone and/or cartilage tissue are recorded and graded according to the following scale: 0: absence, 1: weak, 2: moderate, 3: marked, 4: substantial.

Each of the implants is removed from its implantation site and macroscopic photographs are taken. The size and the weight of the implants are then determined. Each implant is then stored in a buffered 10% formol solution.

Results:

This in vivo experiment makes it possible to measure the osteoinductive effect of BMP-2 by placing the implant in a muscle on the back of a rat. This non-bone site is termed ectopic.

The macroscopic observations of the explants enable us to evaluate the presence of bone tissues and the mass of the implants.

| | | |
|---|---|---|
| Implant | Presence of bone tissues | Mass of implants (mg) |

| | |
|---|---|
| Implant 1 | Implants not found |

| | | |
|---|---|---|
| Implant 2 | 3.6 | 38 |
| Implant 3 | 4.0 | 120 |
| Implant 4 | 2.4 | 84 |
| Implant 5 | 3.8 | 249 |

A dose of 2 μg of BMP-2 in a collagen sponge (implant 1) does not have a sufficient osteoinductive capacity for it to be possible to find collagen implants after 21 days.

A dose of 20 μg of BMP-2 in a collagen sponge (implant 2) results in ossified implants having an average mass of 38 mg being obtained after 21 days.

For the same dose of BMP-2 of approximately 20 μg, the BMP-2/polymer 1 complex (implant 3) in the presence of CaCl₂ in solution in the collagen sponge makes it possible to increase the osteogenic activity of BMP-2. The average mass of the implants 3 is approximately 3 times greater than that of the implants 2.

The lyophilization makes it possible to amplify this gain in osteogenic activity since the average mass of the implants containing 20 μg of BMP-2 in the form of a complex with polymer 1 in the presence of CaCl₂ which are lyophilized in the collagen sponge (implant 5) is twice that of the implants in which the BMP-2/polymer 1 complex in the presence of CaCl₂ is in solution (implant 3).

For a 10-times lower dose of BMP-2, the BMP-2 complex in the presence of CaCl₂ which is lyophilized in the collagen sponge (implant 4) makes it possible to generate ossified implants having double the mass, with a bone score equivalent to those with BMP-2 alone. This new formulation makes it possible to greatly reduce the BMP-2 doses to be administered, while at the same time maintaining the osteogenic activity of this protein.

### EXAMPLE 12

### Preparation of Formulations Containing the rhBMP-2/Polymer 1 Complex

Formulation 5: 552 μl of a solution of rhBMP-2 at 1.35 mg/ml are mixed with 619 μl of a solution of polymer 1 at 60.0 mg/ml. The volume of formulation 5 is made up to 1300 μl by adding sterile water. This solution is left to incubate for two hours at 4° C. and filtered sterilely through 0.22 μm. The concentration of rhBMP-2 in formulation 5 is 0.571 mg/ml and that of polymer 1 is 28.6 mg/ml.

Formulation 6: It is prepared like formulation 5, by mixing 175 μl of a solution of rhBMP-2 at 1.47 mg/ml with 1224 μl of a solution of polymer 1 at 60.0 mg/ml. The volume of formulation 6 is made up to 1800 μl by adding sterile water. The concentration of rhBMP-2 in formulation 6 is 0.14 mg/ml and that of polymer 1 is 40.8 mg/ml.

Formulation 7: It is prepared like formulation 5, by mixing 26.5 μl of a solution of rhBMP-2 at 1.46 mg/ml with 321.7 μl of a solution of polymer 1 at 60.0 mg/ml. The volume of formulation is made up to 772 μl by adding sterile water. The concentration of rhBMP-2 in formulation 7 is 0.05 mg/ml and that of polymer 1 is 25 mg/ml.

### EXAMPLE 13

### Preparation of a Sodium Hyaluronate Gel Containing Calcium Chloride

Gel 1: 10.62 ml of sterile water are introduced into a 50 ml Falcon tube. 0.44 g of sodium hyaluronate (Pharma grade 80, Kibun Food Chemifa, LTD) is added with vigorous stirring on a vortex. 0.14 g of calcium chloride is then added to the sodium hyaluronate gel, also with stirring. The concentration of calcium chloride in the gel is 13.1 mg/ml.

### EXAMPLE 14

### Preparation of a Sodium Hyaluronate Gel Containing the rhBMP-2/Polymer 1 Complex and Calcium Chloride

Gel 2: 1230 μl of formulation 5 are transferred into a sterile 10 ml syringe. 5.8 ml of 4% sodium hyaluronate gel 1 containing calcium chloride at a concentration of 13.1 mg/ml are transferred into a sterile 10 ml syringe. The solution of formulation 5 is added to gel 1 by coupling the two syringes, and the gel obtained is homogenized by passing it from one syringe to the other several times. The opaque gel obtained is transferred into a 50 ml Falcon tube. The concentration of rhBMP-2 in the gel 2 is 0.10 mg/ml and that of polymer 1 is 5.0 mg/ml.

200 μl of gel 2 are injected per implantation site. The dose of rhBMP-2 implanted is 20 μg.

### EXAMPLE 15

### Preparation of a Sodium Hyaluronate Gel Containing the rhBMP-2/Polymer 1 Complex and Calcium Chloride

Gel 3: this gel is prepared as described in example 13, using 1697 μl of formulation 6 and 8 ml of 4% sodium hyaluronate gel containing calcium chloride at a concentration of 15.8 mg/ml. The concentration of rhBMP-2 in gel 3 is 0.025 mg/ml and that of polymer 1 is 7.14 mg/ml.

200 μl of gel 3 are injected per implantation site. The dose of rhBMP-2 implanted is 5 μg.

### EXAMPLE 16

### Preparation of a Sodium Alginate Gel Containing the rhBMP-2/Polymer 1 Complex and Calcium Chloride

Gel 4: this gel is prepared using 772 μl of formulation 7 and 386 μl of sodium alginate gel which is at 40 mg/ml. 40 μl of a solution of calcium chloride at 45.5 mg/ml are added to 60 μl of the sodium alginate gel containing the rhBMP-2/polymer 1 complex. The concentration of rhBMP-2 in gel 4 is 0.02 mg/ml and that of polymer 1 is 10.0 mg/ml.

100 μl of gel 4 are injected per implantation site. The dose of rhBMP-2 implanted is 2 μg.

### EXAMPLE 17

### Preparation of a Collagen Implant Containing a Sodium Alginate Gel Containing the rhBMP-2/Polymer 1 Complex and Calcium Chloride

Implant 6: Gel 5 is prepared using 645 μl of formulation 7 and 323 μl of sodium alginate gel which is at 40 mg/ml. 60 μl of the sodium alginate gel containing the rhBMP-2/polymer 1 complex are added to a Helistat type sterile 200 mm³ crosslinked collagen sponge (Integra LifeSciences, Plainsboro, N.J.). 40 μl of a solution of calcium chloride at 45.5 mg/ml are also added to this sponge. After a contact time of 30 minutes, the sponge is then frozen and lyophilized. This sponge can be directly implanted in the rat.

The dose of rhBMP-2 in implant 1 is 2 μg, that of polymer 1 is 1 mg.

### EXAMPLE 18

### Evaluation of the Osteoinductive Capacity of the Various Formulations

The osteoinductive capacity was evaluated according to the protocol described in example 11.

Results:

This in vivo experiment makes it possible to measure the osteoinductive effect of rhBMP-2 placed in a muscle on the back of a rat. This non-bone site is termed ectopic. The results of the various examples are summarized in the following table.

| | |
|---|---|
| Presence of bone tissue | Mass of explants (mg) |

| | |
|---|---|
| Implant 1 | No explant found |

| | | |
|---|---|---|
| Implant 2 | 3.6 | 38 |
| Gel 2 | 3.7 | 247 |
| Gel 3 | 3.6 | 354 |
| Gel 4 | 2.7 | 63 |
| Implant 6 | 2.4 | 165 |

A dose of 2 μg of rhBMP-2 in a collagen sponge (implant 1) does not have a sufficient osteoinductive capacity for it to be possible to find explants after 21 days.

A dose of 20 μg of rhBMP-2 in a collagen sponge (implant 2) results in ossified explants having an average mass of 38 mg being obtained after 21 days.

For the same rhBMP-2 dose of 20 μg, the sodium hyaluronate gel containing the rhBMP-2/polymer 1 complex (gel 2) in the presence of calcium chloride makes it possible to increase the osteogenic activity of the rhBMP-2. The average mass of the explants obtained with gel 2 is approximately 6 times greater than that of the explants obtained with collagen implants containing 20 μg of rhBMP-2 alone (implant 8).

For an rhBMP-2 dose which is 4 times lower, i.e. 5 μg of rhBMP-2, the rhBMP-2/polymer 1 complex in the presence of CaCl₂ in the sodium hyaluronate gel (gel 3) makes it possible to generate ossified explants having a mass which is 9 times greater, with a bone score equivalent to the explants obtained with the collagen implants containing 20 μg of rhBMP-2 alone (implant 8). This new formulation makes it possible to greatly reduce the doses of BMP-2, while at the same time maintaining the osteogenic activity of this protein.

For an rhBMP-2 dose which is 10 times lower, the rhBMP-2/polymer 1 complex in a sodium alginate gel containing calcium chloride (gel 4) makes it possible to generate ossified explants having a mass which is slightly greater than those obtained with the collagen implants containing 20 μg of rhBMP-2 alone (implant 8). This new formulation makes it possible to greatly reduce the doses of rhBMP-2, while at the same time maintaining the osteogenic activity of this protein.

The alginate gel containing the rhBMP-2/polymer 1 complex can also be placed in a collagen sponge which serves as a support for the growth of the bone cells. In this case also, 2 μg of rhBMP-2 (implant 6) makes it possible to obtain ossified explants having a mass greater than those obtained with the collagen implants containing 20 μg of rhBMP-2 alone (implant 8).

1. Open implant constituted of an osteogenic composition comprising at least:
one osteogenic growth factor,
one soluble salt of a cation at least divalent, and
one organic support,
said organic support comprising no demineralized bone matrix.

2. Implant according to claim 1, wherein the support is constituted of an organic matrix and/or a polymer forming a hydrogel.

3. Implant according to claim 1, wherein the organic matrix is a matrix constituted of crosslinked hydrogels and/or collagen.

4. Implant according to claim 1, wherein the matrix is selected from matrices based on sterilized, purified natural collagen.

5. Implant according to claim 1, wherein the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

6. Implant according to claim 1, wherein the polymer forming a hydrogel, which may be crosslinked or noncrosslinked, is selected from the group of natural polymers, among which are hyaluronic acid, keratan, pullulan, pectin, dextran, cellulose and cellulose derivatives, alginic acid, xanthan, carrageenan, chitosan, chondroitin, collagen, gelatin, polylysine and fibrin, and biologically acceptable salts thereof.

7. Implant according to claim 6, wherein the natural polymer is selected from the group of polysaccharides forming hydrogels, among which are hyaluronic acid, alginic acid, dextran, pullulan, pectin, cellulose and its derivatives, xanthan, carrageenan, chitosan and chondroitin, and biologically acceptable salts thereof.

8. Implant according to claim 6, wherein the natural polymer is selected from the group of polysaccharides forming hydrogels, among which are hyaluronic acid and alginic acid, and biologically acceptable salts thereof.

9. Implant according to claim 1, wherein in that said composition is in the form of a lyophilizate.

10. Implant according to claim 1, wherein the osteogenic growth factor is selected from the group of therapeutically active BMPs (bone morphogenetic proteins).

11. Implant according to claim 1, wherein the osteogenic growth factor is selected from the group constituted of BMP-2 (dibotermin alpha), BMP-4, BMP 7 (eptotermin alpha), BMP-14 and GDF-5.

12. Implant according to claim 1, wherein the osteogenic protein is BMP-2 (dibotermin alpha).

13. Implant according to claim 1, wherein the osteogenic protein is GDF-5.

14. Implant according to claim 1, wherein it further comprises angiogenic growth factors selected from the group constituted of PDGF, VEGF or FGF.

15. Implant according to claim 1, wherein a cation at least divalent is a divalent cation selected from the group constituted of calcium, magnesium or zinc cations.

16. Implant according to claim 1, wherein the soluble divalent-cation salt is a calcium salt, the counterion of which is selected from the chloride, the D gluconate, the formate, the D saccharate, the acetate, the L-lactate, the glutamate, the aspartate, the propionate, the fumarate, the sorbate, the bicarbonate, the bromide or the ascorbate.

17. Implant according to claim 1, wherein the soluble divalent-cation salt is calcium chloride.

18. Implant according to claim 1, wherein the a cation at least divalent is a multivalent cation selected from the group constituted of the cations of iron, aluminum or cationic polymers selected from polylysine, spermine, protamine and fibrin, alone or in combination.

19. Implant according to claim 1, wherein the amphiphilic polysaccharide is selected from the group constituted of polysaccharides functionalized with hydrophobic derivatives.

20. Implant according to claim 1, wherein the amphiphilic polysaccharide is selected from the group constituted of anionic polysaccharides comprising predominantly glycosidic linkages of (1,4), (1,3) and/or (1,2) type, functionalized with at least one tryptophan derivative, corresponding to general formula I below: 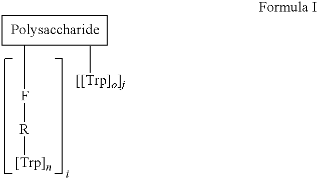
the polysaccharide being constituted predominantly of glycosidic linkages of (1,4) and/or (1,3) and/or (1,2) type,
F resulting from the coupling between the linker arm R and a function —OH of the neutral or anionic polysaccharide, being either an ester function, a thioester function, an amide function, a carbonate function, a carbamate function, an ether function, a thioether function or an amine function,
R being an optionally branched and/or unsaturated chain containing between 1 and 18 carbons, comprising one or more heteroatoms, such as O, N and/or S, and having at least one acid function,
Trp being a residue of an L- or D-tryptophan derivative, produced from the coupling between the amine of the tryptophan derivative and the at least one acid carried by the R group and/or one acid carried by the anionic polysaccharide,
n is the molar fraction of the Trp-substituted Rs and is between 0.05 and 0.7,
o is the molar fraction of the acid functions of the Trp-substituted polysaccharides and is between 0.05 and 0.7,
i is the molar fraction of acid functions carried by the R group per saccharidic unit and is between 0 and 2,
j is the molar fraction of acid functions carried by the anionic polysaccharide per saccharidic unit and is between 0 and 1,
(i+j) is the molar fraction of acid functions per saccharidic unit and is between 0.1 and 2,
when R is not substituted with Trp, then the acid(s) of the R group is (are) a cation carboxylate or cation carboxylates, the cation being a cation of an alkali metal, preferably such as Na or K,
when the polysaccharide is an anionic polysaccharide, when one or more acid function(s) of the polysaccharide is (are) not substituted with Trp, then it (they) is (are) salified with a cation, the cation being an alkali metal cation, preferably such as Na+ or K+, said polysaccharides being amphiphilic at neutral pH.

21. Implant according to claim 1, wherein the amphiphilic polysaccharide is selected from the group constituted of the functionalized anionic polysaccharides of general formula III below: 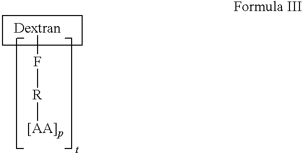
R being an optionally branched and/or unsaturated chain containing between 1 and 18 carbons, comprising one or more heteroatoms, such as O, N and/or S, and having at least one acid function,
F resulting from the coupling between the linker arm R and a function —OH of the neutral or anionic polysaccharide, being either an ester function, a thioester function, an amide function, a carbonate function, a carbamate function, an ether function, a thioether function or an amine function,
AA being a hydrophobic L- or D-amino acid residue produced from the coupling between the amine of the amino acid and an acid carried by the R group, said hydrophobic amino acid being selected from tryptophan derivatives such as tryptophan, tryptophanol, tryptophanamide and 2 indole ethylamine, and the alkali-metal cation salts thereof, or selected from phenylalanine, leucine, isoleucine and valine, and the alcohol, amide or decarboxylated derivatives thereof,
t is the molar fraction of F—R-[AA]n substituent per glycosidic unit and is between 0.1 and 2,
p is the molar fraction of the AA-substituted R groups and is between 0.05 and 1,
when R is not substituted with AA, then the acid(s) of the R group is (are) a cation carboxylate or cation carboxylates, the cation being an alkali metal cation, preferably such as Na+ or K+,
said dextran being amphiphilic at neutral pH.

22. Implant according to claim 1, wherein the amphiphilic polysaccharide is selected from the group constituted of polysaccharides comprising carboxyl functional groups partially substituted with hydrophobic alcohols, of general formula IX: 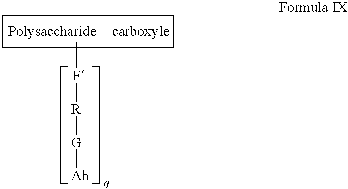
in which q is the molar fraction of the F—R-G-Ah-substituted carboxyl functions of the polysaccharide and is between 0.01 and 0.7,
F′ being an amide function,
G being either an ester function, a thioester function, a carbonate function or a carbamate function,
R being an optionally branched and/or unsaturated chain containing between 1 and 18 carbons, optionally comprising one or more heteroatoms, such as O, N and/or S, and having at least one acid function,
Ah being a residue of a hydrophobic alcohol, produced from the coupling between the hydroxyl function of the hydrophobic alcohol and at least one electrophilic function carried by the R group,
when the carboxyl function of the polysaccharide is not substituted with F′—R-G-Ah, then the carboxyl functional group(s) of the polysaccharide is (are) a cation carboxylate or cation carboxylates, the cation being an alkali metal cation, preferably such as Na+ or K+,
said polysaccharide comprising carboxyl functional groups being amphiphilic at neutral pH.

23. Method for preparing an implant according to the invention, which comprises at least the following steps:
a) providing a solution comprising an osteogenic growth factor,
b) providing an organic matrix and/or a polymer forming a hydrogel,
c) adding the solution containing the growth factor to the organic matrix and/or to the hydrogel, and optionally homogenizing the mixture,
d) adding a solution of a soluble salt of an at least divalent cation to the implant obtained in c),
e) optionally carrying out the lyophilization of the implant obtained in step d).

24. Method according to claim 23, wherein the organic matrix is a matrix constituted of a crosslinked hydrogel and/or collagen.

25. Method according to claim 23, wherein the matrix is selected from matrices based on sterilized, purified natural collagen.

26. Method according to claim 23, wherein the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

27. Method according to claim 23, wherein the polymer forming a hydrogel, which may be crosslinked or noncrosslinked, is selected from the group of natural polymers, among which are hyaluronic acid, keratan, pectin, dextran, cellulose and cellulose derivatives, alginic acid, xanthan, carrageenan, chitosan, chondroitin, collagen, gelatin, polylysine and fibrin, and biologically acceptable salts thereof.

28. Method according to claim 23, wherein the natural polymer is selected from the group of polysaccharides forming hydrogels, constituted of hyaluronic acid, alginic acid, dextran, pectin, cellulose and its derivatives, pullulan, xanthan, carrageenan, chitosan and chondroitin, and biologically acceptable salts thereof.

29. Method according to claim 23, wherein the natural polymer is selected from the group of polysaccharides forming hydrogels, constituted of hyaluronic acid and alginic acid, and biologically acceptable salts thereof.

30. Method according to claim 23, wherein the solution of a soluble salt of a cation at least divalent is a divalent-cation solution.

31. Method according to claim 30, wherein the soluble divalent-cation salt is selected from magnesium salts, the counterion of which is the chloride, the D gluconate, the formate, the D saccharate, the acetate, the L-lactate, the glutamate, the aspartate, the propionate, the fumarate, the sorbate, the bicarbonate, the bromide or the ascorbate.

32. Method according to claim 31, wherein the soluble divalent-cation salt is selected from the chloride, the D gluconate, the formate, the D saccharate, the acetate, the L-lactate, the glutamate, the aspartate, the propionate, the fumarate, the sorbate, the bicarbonate, the bromide or the ascorbate.

33. Method according to claim 31, wherein in step d), the soluble divalent-cation salt is calcium chloride.

34. Method according to claim 23, wherein in step a), a solution of a nonosteogenic growth factor is also provided.
